(19) United States
(12) Patent Application Publication
(10) Pub. No.: US 2023/0059693 A1
(43) Pub. Date: Feb. 23, 2023

(54) DATA ANALYSIS SYSTEM AND DATA ANALYSIS METHOD

(71) Applicant: Wistron Corp., New Taipei City (TW)

(72) Inventors: Po-Chia LIAO, New Taipei City (TW); Yi-Han LIN, New Taipei City (TW); Ming-Lun WU, New Taipei City (TW); Wen-Hsin HU, New Taipei City (TW)

(21) Appl. No.: 17/517,582

(22) Filed: Nov. 2, 2021

(30) Foreign Application Priority Data
Aug. 23, 2021 (TW) 110131023

(51) Int. Cl. G16H 15/00; G16H 10/60; G16H 20/00; G06F 40/103

(52) U.S. Cl. CPC G16H 15/00; G16H 10/60; G16H 20/00; G06F 40/103

(57) ABSTRACT

A data analysis method is provided to optimize the content of the medical record, and input the optimized medical record report into an application model, so that the application model can link the medical record report with the diagnosis code, and output an accurate recommended diagnosis code. With the assistance of the application model for the search of diagnostic codes, the overall quality of medical care is further improved.

### CROSS REFERENCE TO RELATED APPLICATIONS

This application claims priority of Taiwan Patent Application No. 110131023, filed on Aug. 23, 2021, the entirety of which is incorporated by reference herein.

### BACKGROUND OF THE INVENTION

### Field of the Invention

The present disclosure relates to a data analysis system and a data analysis method and, in particular, to a data analysis system and a data analysis method applied to optimize data and data visualization.

### Description of the Related Art

Disease classification is a classification system that categorizes the affected body or disease group according to established criteria. The purpose of the International Classification of Diseases is to systematically record, analyze, interpret, and compare morbidity or death data collected in different countries and regions, and at different times.

International Classification of Disease (ICD) is used to translate the diagnosis of diseases and other health problems from text into English letters and numbers mixed configuration decoding or alphanumeric code to facilitate data access and analysis. The first three codes are the core classification codes, which are the international notifications of the World Health Organization (WHO) cause of death database and the decoding of the more internationally necessary classification items; the last four codes are the detailed classification items. Since the 10th edition of ICD (ICD-10 for short) was passed by WHO in 1989, one after another all countries have adopted it for use online.

However, the structure and characteristics of the disease codes from ICD-9 to ICD-10 have changed, the disease diagnosis codes are completely different, and the complexity and precision have been greatly improved. Therefore, the number has also been revised from the original 13,000 to 68,000. Doctors and clinical staff need to relearn and adapt, which also adds administrative inconvenience to the complicated clinical work. Doctors are responsible for clinical, teaching, administrative, and research tasks. However, due to compliance with national health policies or health insurance application and payment specifications, writing medical records takes up a lot of time for physicians and shortens the time available to care for patients.

Therefore, how to automatically optimize medical record data written by doctors and present the optimized data in a better visual manner has become one of the problems that need to be solved in this field.

### BRIEF SUMMARY OF THE INVENTION

In accordance with one feature of the present disclosure, the present disclosure provides a data analysis system. The data analysis system includes an electronic device and a processor. The electronic device, configured to receive a part of contents of a plurality of medical information fields. The processor is configured to generate an optimization report based on the part of the contents of the medical information fields. The processor inputs the optimization report into an application model. The application model outputs a plurality of diagnostic codes corresponding to the optimization report. The processor generates a heat map according to a plurality of weights corresponding to a plurality of words in the optimization report, and the processor displays the heat map through a user interface of the electronic device.

In accordance with one feature of the present disclosure, the present disclosure provides a data analysis method. The data analysis method includes following steps. A user interface is displayed. The user interface includes a plurality of medical information fields. A part of the contents of the medical information fields is transmitted. A processor generates an optimization report based on the part of the contents of the medical information fields. The optimization report is input into an application model by the processor. The application model outputs a plurality of diagnostic codes that correspond to the optimization report. A heat map is generated by the processor according to a weights corresponding to a words in the optimization report. The heat map is displayed through a user interface by the processor of the electronic device.

In summary, the data analysis system and data analysis method can assist physicians in writing medical records with the assistance of abbreviation reduction and typo-correction suggestions, so as to optimize the medical record report, and input the optimized medical record report into an application model to enable the application model to link the medical record report with the diagnosis code and output accurate recommended diagnosis codes. With the aid of the application model for the diagnosis code search, medical staff can spend more time studying the medical records, including the examinations performed by the patient, whether the symptoms are fully reflected in the diagnosis, whether there are missing data, and how to do it without violating the medical principles according to the corresponding cost data of the corresponding candidate diagnosis codes. The health insurance payment is improved, and the overall quality of the medical treatment is further improved.

### BRIEF DESCRIPTION OF THE DRAWINGS

In order to describe the manner in which the above-recited and other advantages and features of the disclosure can be obtained, a more particular description of the principles briefly described above will be rendered by reference to specific examples thereof which are illustrated in the appended drawings. Understanding that these drawings depict only exemplary aspects of the disclosure and are not therefore to be considered to be limiting of its scope, the principles herein are described and explained with additional specificity and detail through the use of the accompanying drawings in which:

FIG. 1 is a block diagram of a data analysis system 100 in accordance with one embodiment of the present disclosure.

FIG. 2 is a flowchart of a data analysis method 200 in accordance with one embodiment of the present disclosure.

FIG. 3 is a schematic diagram of a user interface in accordance with one embodiment of the present disclosure.

FIG. 4 is a schematic diagram of an application model 18 in accordance with one embodiment of the present disclosure.

FIG. 5 is a schematic diagram of a heat map in accordance with one embodiment of the present disclosure.

FIG. 6 is a schematic diagram of a data analysis system applied to an outpatient or emergency situation in accordance with one embodiment of the present disclosure.

FIG. 7 is a schematic diagram of a data analysis system applied to a patient's hospitalization situation in accordance with one embodiment of the present disclosure.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present invention will be described with respect to particular embodiments and with reference to certain drawings, but the invention is not limited thereto and is only limited by the claims. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

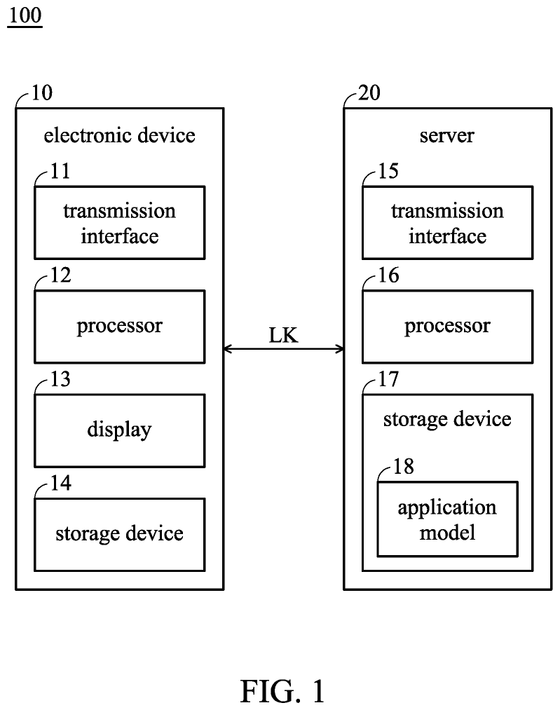

Please refer to FIG. 1, FIG. 1 is a block diagram of a data analysis system 100 in accordance with one embodiment of the present disclosure. The data analysis system 100 includes an electronic device 10 and a server 20. In one embodiment, the electronic device 10 includes a transmission interface 11, a processor 12, a display 13 and a storage device 14. In one embodiment, the server 20 includes a transmission interface 15, a processor 16 and a storage device 17. In one embodiment, the electronic device 10 establishes a communication connection LK with the server 20 through a wired or wireless method.

In one embodiment, the processor 16 in the server 20 accesses and executes programs stored in the storage device 17 to implement an application model 18. In one embodiment, the application model 18 is implemented by software or firmware. In one embodiment, the application model 18 is implemented by a hardware circuit. For example, the application model 18 may be composed of active components (such as switches, transistors) and passive components (such as resistors, capacitors, and inductors), and its hardware circuit is coupled to the processor 16. In one embodiment, the processor 16 is used to access the operation result of the application model 18. In an example, after the processor 16 performs further calculations on the calculation results, the further calculation results can be stored back to the storage device 17. In one embodiment, the processor 16 is used to access the operation result of the application model 18. In an example, after the processor 16 performs further calculations on the calculation results, the further calculation results can be stored back to the storage device 17.

In one embodiment, each of the storage device 14, the storage device 17 can be implemented as a read-only memory, flash memory, floppy disk, hard disk, optical disk, flash drive, tape, a database that can be accessed by the network, or those familiar with this technique can easily think of storage media with the same functions.

In one embodiment, each of the processor 12 and the processor 16 can be implemented by a volume circuit such as a micro controller, a microprocessor, a digital signal processor (DSP), and on-site programmable logic. It is implemented by a Field Programmable Gate Array (FPGA), an Application Specific Integrated Circuit (ASIC) or a logic circuit.

In one embodiment, the transmission interfaces 11, 15 can be Wi-Fi devices, Bluetooth devices, wireless network interface cards, or other devices for transmitting data.

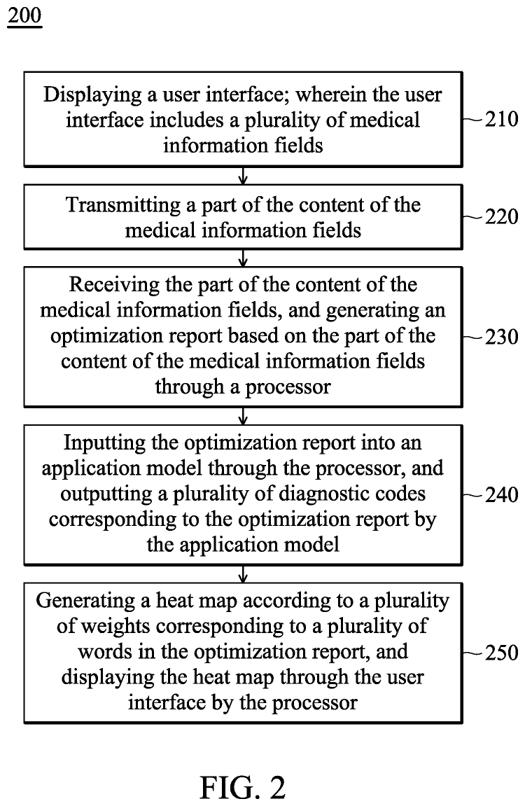

Please refer to FIG. 2. FIG. 2 is a flowchart of a data analysis method 200 in accordance with one embodiment of the present disclosure. The data analysis method 200 can be implemented by the components shown in FIG. 1.

In step 210, the electronic device 10 is used to display a user interface, and the user interface includes a plurality of medical information fields.

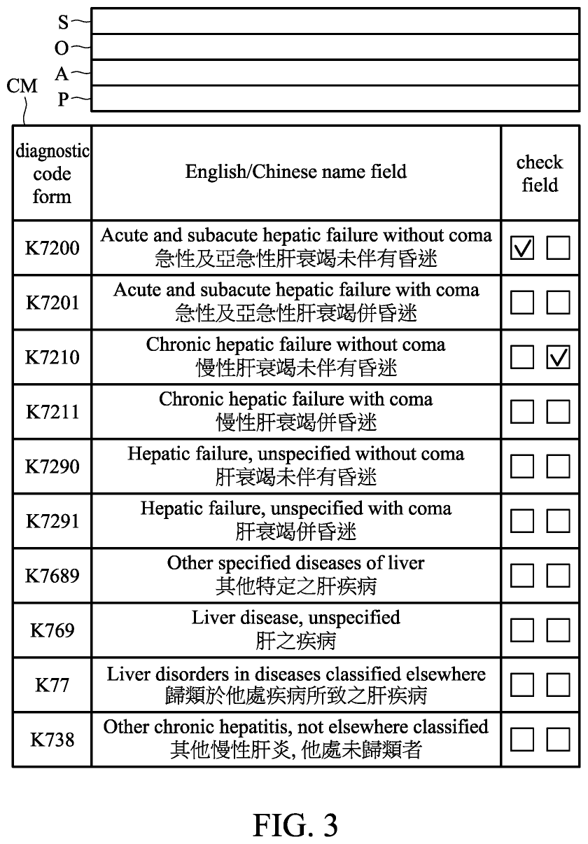

Please refer to FIG. 3, FIG. 3 is a schematic diagram of a user interface in accordance with one embodiment of the present disclosure. In an embodiment, the electronic device 10 may be a mobile phone, a tablet, a laptop, or a desk phone. The electronic device 10 is generally placed in a hospital. The electronic device 10 can be mounted or communicatively connected to the Hospital Information System (HIS). Hospital information system refers to the use of modern computer software technology and network communication technology to realize the comprehensive management of the flow of people, logistics, and finances in the hospital. The user interface may be one of the pages in the hospital information system, and the user interface is used for medical staff to input medical record related information.

In one embodiment, the user interface displayed on the display 13 of the electronic device 10 includes a plurality of medical information fields. These medical information fields include, for example, a subjective field S and a diagnosis observation field O, a diagnosis assessment field A, and a treatment plan field P. Each field contains the content of the patient's subjective complaint, the content of the diagnosis and observation, the content of the diagnosis and evaluation, and the content of the treatment plan. In another embodiment, the content of the patient's subjective complaint, the content of diagnosis and observation, the content of diagnosis and evaluation, and the content of the treatment plan are displayed on the display 13 of the electronic device 10. The content of the treatment plan is combined or scattered in the patient's medical record. The embodiment of the present invention does not limit the presentation form of each field or the content corresponding to the fields.

The content of subjective field S is the patient's symptoms. The patient's conscious symptoms include the patient's main complaint, symptoms, time of onset, current medical history, past medical history, and personal history. For example, recording the patient's statement: the right lower abdominal pain began yesterday afternoon, and the fever began to reach 38.5 degrees Celsius at night. This has not happened in the past, and there are no chronic diseases.

The content of the diagnosis observation field O is the doctor's examination findings, including examination findings and various examination reports, for example, records that the doctor observes: the patient has pain near the belly button, vomiting, pressure pain in the right lower abdomen, leukocytosis, etc.

The content of the diagnosis assessment field A is diagnostic evaluation, that is, diagnosis or impression. For example, the content of the diagnosis assessment field A record: the patient may suffer from appendicitis.

The content of treatment plan field P is a treatment plan, including various treatments or prescriptions, such as appendectomy. In addition, multiple medical information fields are further divided into medical information fields related to the outpatient model and medical information fields related to the inpatient model. The content of the medical information field of the hospitalization model contains the rest of the patient's text reports (consultation, pathology, surgery, examination) within six months. The medical information field of the outpatient model includes at least one of subjective field S and a diagnosis observation field O, a diagnosis assessment field A, and a treatment plan field P. The electronic device 10 fills in or substitutes the content of the medical information field related to the current patient.

In step 220, the electronic device 10 transmits a part of the content of the medical information fields.

In one embodiment, the contents of the medical information field transmitted by the electronic device 10 through the transmission interface 11 include the content of a subjective field (for example, the content of subjective field S), and the content of a diagnosis observation field (for example, the content of diagnosis observation field O), and the content of a diagnosis assessment field (for example, the content of diagnosis assessment field A).

In step 230, the transmission interface 15 of the server 20 receives the part of the content of the medical information fields, and generates an optimization report based on the part of the content of the medical information fields through a processor 16.

In one embodiment, the content of the medical information field received by the server 20 through the transmission interface 15 includes the content of a subjective field, the content of a diagnosis observation field, and the content of a diagnosis assessment field.

In one embodiment, the server 20 uses the processor 16 to perform a content optimization based on a part of the contents of the multiple medical information fields to generate an optimization report.

In one embodiment, the processor 16 of the server 20 optimizes the content of a subjective field, the content of a diagnosis observation field, and the content of a diagnosis assessment field.

In one embodiment, the content optimization includes using an abbreviation reduction Application Programming Interface (API) to change the abbreviations in a part of the contents of the medical information fields to full names. Moreover, at least a part of the contents of the medical information fields is automatically changed to correct text through a typo-correction suggestion application program interface, so as to automatically change the typo to the correct word or receive a corrected word that corrects the typo, to generate the optimization report.

In one embodiment, content optimization includes changing the content of a subjective field, the content of a diagnosis observation field, and the content of a diagnosis assessment field through an abbreviation recovery application interface, respectively, to make the abbreviations in the content of a subjective field, the content of a diagnosis observation field, and the content of a diagnosis assessment field into full name.

In one embodiment, the content of a subjective field, the content of a diagnosis observation field, and the content of a diagnosis assessment field each use a typo-correction suggestion application program interface to automatically change the typo into the correct one or receive suggested text to correct the typo to generate the optimization report.

For example, the server 20 sends a text containing the content of a subjective field, the content of a diagnosis observation field, and the content of a diagnosis assessment field to the electronic device 10. The text provides some candidate words for uncertain words (such as typos, abbreviations) for doctors to choose. After the doctor confirms that the content of the text is complete and correct, the electronic device 10 sends the text back to the server 20, and the text at this time is the optimization report.

Since each doctor has his/her own different writing style for medical records, doctors often use disease abbreviations in the medical records to record. However, the abbreviation habits of each department or each doctor are different, and the divergence is great. At the same time, doctors face busy clinical work and have limited time to write medical orders, and often some typos can be found in the text content of the medical records. If according to the content of the medical records written by the doctor, the corresponding tenth edition of the International Classification of Disease (ICD), later called the ICD-10 code, is output through the application model 18, thereby reducing the workload of the hospital's disease classifiers. The content quality of written medical records is very important.

Therefore, through step 230, the doctor can assist the doctor with abbreviation reduction and typo-correction suggestions when writing the medical record, so that the doctor can produce an optimized medical record report with high quality content (i.e., optimization report) in a limited time, and avoid being returned and re-editing the documents. Moreover, the high-quality medical records improved the accuracy of the application model 18. In one embodiment, the server 20 transmits the optimized text of the content of a subjective field, the content of a diagnosis observation field, and the content of a diagnosis assessment field to the electronic device 10. The electronic device 10 displays the optimized medical record report (i.e., optimization report) on the display 13, or updates the content in each field to the optimized content.

In step 240, the server 20 inputs the optimization report into an application model 18 through the processor 16, and the application model 18 outputs a plurality of diagnostic codes corresponding to the optimization report.

In an embodiment, the diagnostic codes corresponding to the optimized report output by the application model comply with a disease classification coding rule of the tenth edition of the International Statistical Classification of Diseases (ICD-10). The disease classification coding rule is for multiple disease diagnoses and multiple predictions, and more than 60,000 diagnostic codes corresponding to these diagnoses and these predictions are compiled.

In one embodiment, the application model 18 is implemented by a Bidirectional Encoder Representations from Transformers-Convolutional Neural Networks (BERT-CNN), hereinafter referred to as BERT-CNN. However, this is an example, and the application model 18 can be implemented by other convolutional neural networks capable of generating vocabulary vectors or weights.

Please refer to the diagnostic code form CM in FIG. 3. When the server 20 uses the processor 16 to input the optimization report into the application model 18 (for example, BERT-CNN), the application model 18 outputs multiple diagnostic codes corresponding to the optimization report. These diagnostic codes represent that based on the optimization report, the application model 18 outputs the diagnosis results related to the optimization report. In one embodiment, the server 20 transmits these diagnostic codes to the electronic device 10 and displays the diagnostic code form CM corresponding to the diagnostic codes on the display 13.

Since the description of the diagnosis result (such as the English/Chinese name field) is relatively lengthy, doctors who are proficient in the ICD-10 diagnosis code can quickly check one or more diagnosis results that the patient matches through the diagnosis code. On the other hand, doctors who are not yet familiar with the ICD-10 diagnosis code can still check one or more diagnosis results that the patient matches through the English/Chinese name field.

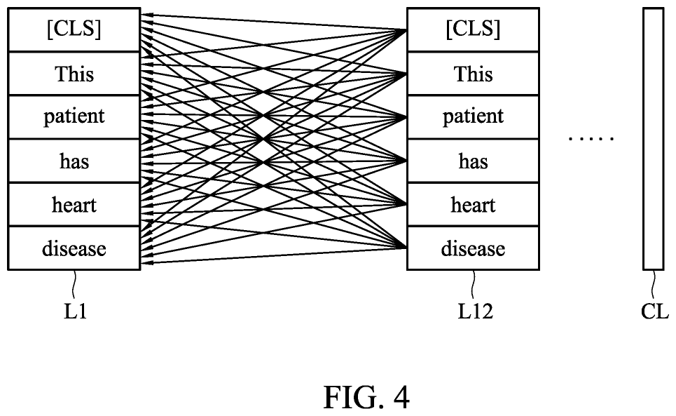

Please refer to FIG. 4, FIG. 4 is a schematic diagram of an application model 18 in accordance with one embodiment of the present disclosure. FIG. 4 is a schematic diagram of an application model 18 according to an embodiment of the present invention. The application model 18 in FIG. 4 uses the BERT-CNN architecture. BERT-CNN is a two-stage transfer learning of state-of-the-art (SOTA) in the field of Natural Language Processing (NLP) in recent years, the two-stage respectively: pre-training and fine-tuning.

In the pre-training stage, a large number of textual materials (such as the content of the patient's subjective complaint, the content of diagnosis and observation, the content of diagnosis and evaluation, and the content of the treatment plan, medical and biotechnology-related papers, newspapers, journals) related to medical and biotechnology are used in advance to train a language model (i.e., application model 18) in an unsupervised learning manner.

In the fine-tuning stage, it is aimed at the classification task of diagnostic codes. It uses class-labeled data for training the application model 18 and performs supervised learning on the application model 18 to fine-tune the parameters, and then make predictions on new data. The class label is the ICD-10 code. Through this training method, the application model 18 can understand the content relationship of the context in the medical record. The application model 18 learns from the description of the patient's condition and patient history written by the doctor. Moreover, the application model 18 is trained with medical knowledge, which accurately establishes the link between the medical record and the diagnosis code, and accurately recommends the diagnosis code.

Self-attention is an important mechanism for the implementation of clinical BERT-CNN training application model 18. Take “This patient has heart disease” as an example, when performing Self-Attention, there are the following steps: (1) in the classification task, use the processor 16 or manually insert the prediction label “[CLS]” symbol at the beginning of each sentence (as indicated in the first column of the conversion layers L1 and L12 in FIG. 4). The purpose of the self-attention mechanism is to understand the meaning of the text and predict the corresponding category (for example, the category is an ICD-10 diagnostic code). This mechanism is fixed at the forefront of the text, using the processor 16 or manually adding the tag “[CLS]” as the basis for subsequent forecasts.

(2) Convert each vocabulary into word embedding: this step will convert all vocabularies into vectors of the same dimension (each model architecture will have different dimensions, Clinical BERT has 768 dimensions), and each vocabulary has a different dimension. The vectors are all different, and the application model 18 defines the vector values of these words in advance.

(3) Update the word embedding of each vocabulary according to the context: each vocabulary needs to undergo 12 conversions in the application model 18 (in this example, 12 transformation layers (transformer layers) L1 to L12 are taken as an example). Each layer accepts a set of word vectors as input, and produces the same number of word vectors as output. After each conversion, a different word vector will be obtained. The application model 18 refers to the content of the context to determine the value of the converted vector. Moreover, according to different context semantics, the reference weights are also different, and the application model 18 automatically adjusts these weights during the learning process. In one embodiment, after all characters are converted 12 times, the prediction label “[CLS]” is used to predict the output of the last layer of conversion. Only the first vector (corresponding to the “[CLS]” symbol) will be input to the classifier, and the “[CLS]” vector will be used to predict the ICD-10 diagnostic code using the Linear Regression classification method. In the self-attention prediction mechanism, the application model 18 adjusts the weight of the reference according to the content of the context. Since the prediction is based on the vector of the “[CLS]” label, by observing the weight value referenced by “[CLS]”, it can be understood “which words are the main references when the model performs predictions”.

Take FIG. 4 as an example, the final “[CLS]” will get 6 weights, these weights are “[CLS]” tags refer to the weight of “[CLS]”, “This”, “patient”, “has”, “Heart”, and “disease”. As shown in Table 1 below:

TABLE 1

| word | [CLS] | This | patient | has | heart | Disease |
|---|---|---|---|---|---|---|
| weight | 0. 1 | 0. 1 | 0.2 | 0.05 | 0.9 | 0.56 |

By visualizing these weight values, the heavier the weight, the darker the color will be drawn, and vice versa. Then the feature extraction can be performed on the focus of the model prediction, and the heat map visualization results can be obtained. This will be described in detail in step 250.

In other words, as shown in Table 1 and FIG. 4, BERT-CNN determines multiple word vectors based on the context of the content of the optimization report, and the processor 16 performs processing based on multiple word features defined in advance in each layer of BERT-CNN. Feature extraction is used to extract these words. After these word vectors pass through a classification layer CL of BERT-CNN, the classification layer CL outputs the corresponding weights for each word vector.

In one embodiment, after the processor 16 of the server 20 inputs the content of the patient's subjective complaint, the content of diagnosis and observation, the content of diagnosis and evaluation, and the content of the treatment plan into the BERT-CNN, multiple diagnostic codes (for example, ICD-10 diagnostic code) about the content are obtained. The processor 16 sorts the diagnostic codes corresponding to the weights according to the weights in descending order to generate a diagnosis code list, and selects a certain number of diagnostic codes (for example, the top ten) for providing them to the doctor for reference.

In step 250, the processor 16 generates a heat map according to a plurality of weights corresponding to a plurality of words in the optimization report, and the processor 16 displays the heat map through the user interface.

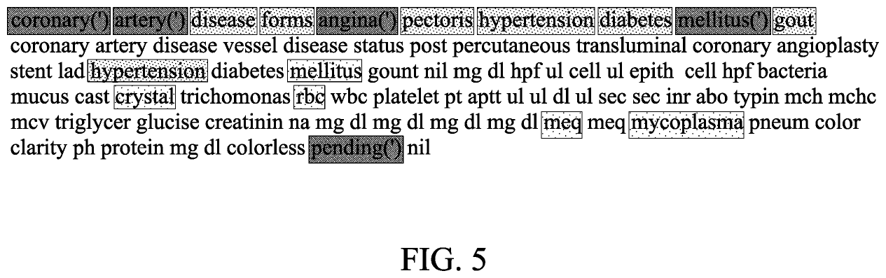

Please refer to FIG. 5. FIG. 5 is a schematic diagram of a heat map in accordance with one embodiment of the present disclosure. As shown in FIG. 5, the processor 16 marks the words corresponding to the weights in different colors in the optimization report to generate a heat map. For example, words with higher weights are marked with darker colors, and words with lower weights are marked with lighter colors. In one embodiment, the depth to the shallowness of the color of the weights are corresponded according to the weights from large to small.

In this way, readers (such as doctors) do not read all articles (such as subjective field S and a diagnosis observation field O, a diagnosis assessment field A, and a treatment plan field P) can quickly focus on the main content of a large number of articles (medical history-related articles) by visually marking the color of words without reading all the articles.

In one embodiment, the processor 16 is further used to generate a word cloud based on these weights. The word cloud is a combination of various words to form a cloud-like graphic. The purpose of the word cloud is to allow readers to quickly focus on the main content of a large number of articles (for example, the most weighted vocabulary, the largest and most obvious font in the word cloud) without reading all the articles.

From the above steps, through extensive collection of the hospital's past outpatient, emergency and inpatient diagnosis results, the content includes the ICD-10 diagnosis code of each patient and the subjective and objective description of the outpatient and emergency department, or the disease extraction and course records during the hospitalization process, etc. The content of the written doctor's order, as well as the patient's examination, surgery, consultation and pathology text report, these data are input into the application model 18, and the application model 18 performs the classification recommendation of the ICD-10 diagnosis code.

Because the content of the patient's subjective complaint, the content of diagnosis and observation, the content of diagnosis and evaluation, and the content of the treatment plan entered by the doctor during the outpatient and emergency consultation, and the admission note, the progress note, and the discharge summary written by the doctor for the inpatient when the patient is hospitalized, are quite different in text structure and content. Therefore, when the application model 18 is used for training, the model is trained separately according to the different data sources of the use situation to ensure the recommendation quality of the diagnostic code classification.

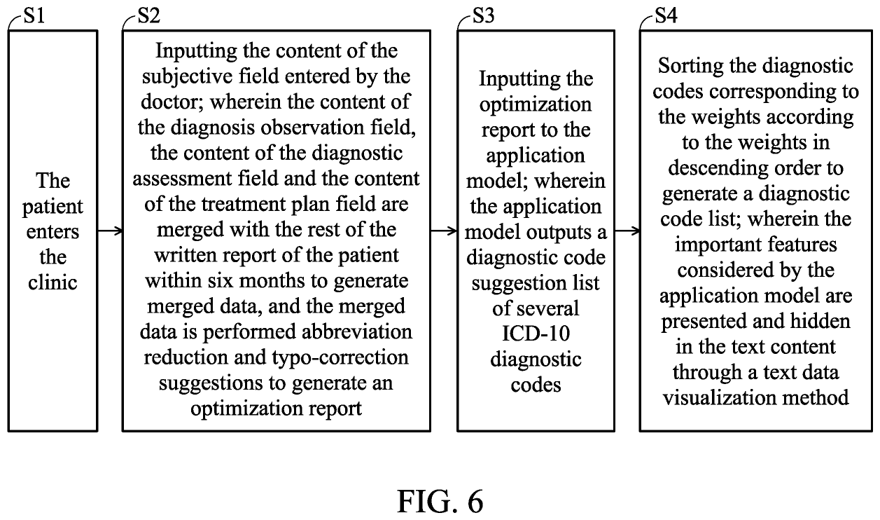

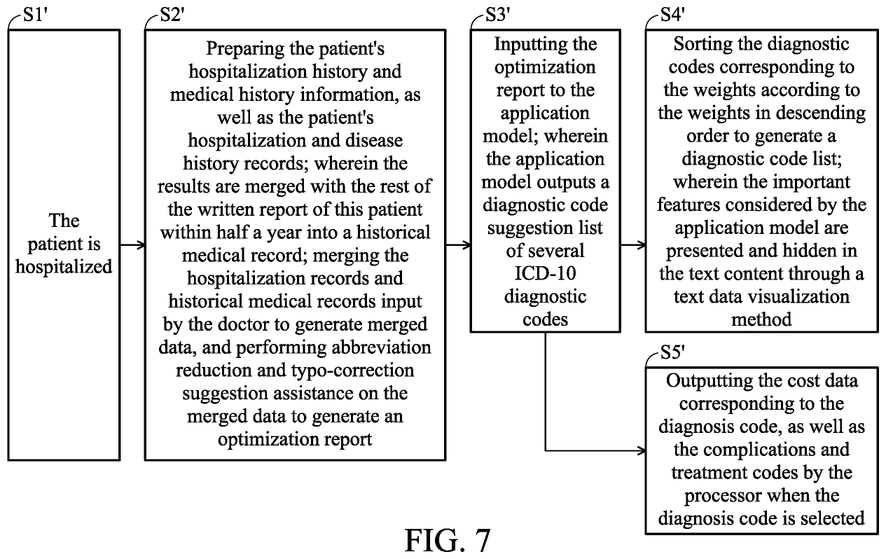

Please refer to FIGS. 6-7. FIG. 6 is a schematic diagram of a data analysis system applied to an outpatient or emergency situation in accordance with one embodiment of the present disclosure. FIG. 7 is a schematic diagram of a data analysis system applied to a patient's hospitalization situation in accordance with one embodiment of the present disclosure.

In one embodiment, in an outpatient or emergency situation (as shown in FIG. 6), the patient enters the clinic (step S1), and the processor 12 immediately inputs the content of the subjective field S entered by the doctor (for example, the patient says he has a sore throat and keeps vomiting), the content of the diagnosis observation field O (for example, the doctor observes that the patient has a fever and abnormal blood pressure), the content of the diagnostic assessment field A (for example, the doctor judges food poisoning and/or gastroenteritis) and the content of the treatment plan field P (such as medication and/or hospitalization observation) are merged with the rest of the written report (consultation, pathology, surgery, examination) of the patient within six months to generate merged data, and the merged data is performed abbreviation reduction and typo-correction suggestions to generate an optimization report (step S2), and then transmit the optimization report to the server 20 through the transmission interface 11. The processor 16 inputs the optimization report to the application model 18, and the application model 18 outputs a diagnostic code suggestion list of several ICD-10 diagnostic codes (step S3). The processor 16 sorts the diagnostic codes corresponding to the weights according to the weights in descending order to generate a diagnostic code list. For example, the processor 16 provides the top 10 most likely ICD-10 diagnostic codes for doctors or disease analysts as reference. The important features considered by the application model 18 are presented and hidden in the text content through a text data visualization method (such as labeling vocabulary color according to weight, word cloud) (step S4).

In one embodiment, in a situation where the patient has been hospitalized (as shown in FIG. 7), after the patient is hospitalized (step S1′), the hospital information system prepares the patient's hospitalization history and medical history information, as well as the patient's hospitalization and disease history records. The results are merged with the rest of the written report (consultation, pathology, surgery, examination) of this patient within half a year into a historical medical record. The processor 12 merges the hospitalization records and historical medical records input by the doctor to generate merged data. Moreover, the processor 12 performs abbreviation reduction and typo-correction suggestion assistance on the merged data to generate an optimization report (step S2′), and then transmits the optimization report through the transmission interface 11 to the server 20. The processor 16 inputs the optimization report to the application model 18. Moreover, the application model 18 outputs a diagnostic code suggestion list of several ICD-10 diagnostic codes (step S3′). The processor 16 sorts the diagnostic codes corresponding to the weights according to the weights in descending order to generate a diagnostic code list. For example, the processor 16 provides the top 10 most likely ICD-10 diagnostic codes for doctors or diagnosticians as reference. The important features considered by the application model 18 are presented and hidden in the text content through a text data visualization method (such as labeling vocabulary color according to weight, word cloud) (step S4′). On the other hand, after the completion of step S3′, when the diagnosis code is selected (for example, the doctor selects the diagnosis code), the processor 16 outputs the cost data corresponding to the diagnosis code, as well as the complications and treatment codes, to prompt information for the doctor to choose (Step S5′).

In one embodiment, the doctor checks multiple options in the diagnostic code form CM (the selected options are regarded as candidate diagnostic codes), thereby giving the following instructions to the processor 16 to make the processor 16 select the multiple candidate diagnosis codes in the diagnostic code form CM. The processor 16 receives treatment data corresponding to each of the candidate diagnosis codes, and these treatment data are each recorded in a treatment plan field P.

In one embodiment, the processing data comes from the history records stored in the storage device 17 of the server 20 or the storage device 14 of the electronic device. Each diagnosis code (for example, the diagnosis code for gastroenteritis) corresponds to at least one treatment data (for example, prescription, hospital observation, and infusion).

In one embodiment, the processor 16 selects a plurality of candidate diagnostic codes in the diagnostic code list and generates a corresponding cost data corresponding to each of the candidate diagnostic codes according to a history record, and each of these cost data is recorded in a cost field corresponding to these candidate diagnostic codes.

In one embodiment, in response to the processor 16 receiving treatment data corresponding to each of these candidate diagnostic codes, the processor 16 generates corresponding cost data corresponding to each of these candidate diagnostic codes according to the corresponding treatment data or historical records. These cost data are respectively recorded in the cost field.

In one embodiment, the data analysis system and data analysis method are used for data analysis. The time range is from January 2016 to February 2020. There are 3,112,158 consultation data in outpatient and emergency departments, and the ICD-10 diagnosis code covers 12,732 different categories. A total of 83,441 hospitalization data were hospitalized, and the ICD-10 diagnostic code covers 3,772 different types of diagnostic codes. In order to avoid over-fitting and improve the generalization ability of the model, the data is divided by time, and the data from 2016 to 2019 are used as the training set. The data from January to February 2020 is used as a test set to verify the accuracy of the application model 18. The accuracy of the first ten predicted diagnosis codes of the main diagnosis verified by the outpatient and emergency models using the test set is 91.45%. The hospitalization model uses the test set to verify that the accuracy of the first ten diagnosis codes of the main diagnosis is 89.35%. The accuracy is calculated as the coupling rate between the main diagnosis of the test set and the ten diagnosis codes predicted by the model (the number of samples of the main diagnosis of the test set in the ten predicted diagnosis codes/the total number of samples in the test set).

In addition, the above-mentioned application model 18 uses a large amount of labeled data for fine-tuning training, so the number of diagnostic code categories currently predictable by the application model 18 can be forwarded as the range covered by the sample data. Through the continuous provision of the collected data in the future, the increase in the amount of data can continue to be used for learning and correction for the application model 18, and the range of predictable diagnostic code categories will also increase, and the performance of the application model 18 will continue to improve. In turn, the accuracy of the forecast is improved.

In summary, the data analysis system and data analysis method can assist physicians in writing medical records with the assistance of abbreviation reduction and typo-correction suggestions, so as to optimize the medical record report, and input the optimized medical record report into an application model to make the application model able to link the medical record report with the diagnosis code and output accurate recommended diagnosis codes. With the aid of the application model for the diagnosis code search, medical staff can spend more time studying the medical records, including the examinations performed by the patient, whether the symptoms are fully reflected in the diagnosis, whether there are missing data, and how to do it without violating the medical principles according to the corresponding cost data of the corresponding candidate diagnosis codes. The health insurance payment is improved, and the overall quality of the medical treatment is further improved.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such a feature may be combined with one or more other features of the other implementations as may be desired and advantageous for any given or particular application.

What is claimed is:

1. A data analysis system, comprising:
an electronic device, configured to receive a part of contents of a plurality of medical information fields; and
a processor, configured to generate an optimization report based on the part of the contents of the medical information fields;
wherein the processor inputs the optimization report into an application model, the application model outputs a plurality of diagnostic codes corresponding to the optimization report, and the processor generates a heat map according to a plurality of weights corresponding to a plurality of words in the optimization report, and the processor displays the heat map through a user interface of the electronic device.

2. The data analysis system of claim 1, wherein the electronic device displays the user interface, the user interface comprises the medical information fields, and the electronic device transmits the part of the contents of the medical information fields through a first transmission interface, and the data analysis system further comprises:
a server, configured to receive the part of the contents of the medical information fields through a second transmission interface;
wherein the processor is located in the server;
wherein the medical information fields include a subjective field, a diagnosis observation field, a diagnosis assessment field, and a treatment plan field; wherein the contents of the part of the fields includes the contents of the subjective field, the contents of the diagnosis observation field, and the contents of the diagnosis assessment field, as well as the rest of the text report of the patient within half a year.

3. The data analysis system of claim 2, wherein the server performs a content optimization based on the part of the contents of the medical information fields through the processor to generate the optimization report.

4. The data analysis system of claim 3, wherein the content optimization includes using an abbreviation reduction Application Programming Interface (API) to change abbreviations in the part of the contents of the medical information fields to full names; wherein the part of the contents of the medical information fields is automatically changed to correct text through a typo-correction suggestion application program interface, so as to automatically change a typo to the correct word or receive a corrected word that corrects the typo, to generate the optimization report.

5. The data analysis system of claim 1, wherein the application model outputs corresponding to the diagnostic codes;
wherein the diagnostic codes comply with a disease classification coding rule of the tenth edition of the International Classification of Disease (ICD);
wherein for multiple disease diagnosis and multiple predictions, the disease classification coding rule compiles the diagnostic codes corresponding to these disease diagnoses and the diagnostic codes for these predictions.

6. The data analysis system of claim 1, wherein the processor sorts the diagnostic codes corresponding to the weights according to the weights in descending order to generate a diagnosis code list.

7. The data analysis system of claim 6, wherein the processor selects a plurality of candidate diagnosis codes in the diagnosis code list, receives treatment data corresponding to each of the candidate diagnosis codes, and records the treatment data in a treatment plan field.

8. The data analysis system of claim 6, wherein the processor selects a plurality of candidate diagnostic codes in the diagnosis code list, and based on a historical record, generates cost data corresponding to each of the candidate diagnostic codes, and records the cost data respectively in a cost field corresponding to the candidate diagnosis codes.

9. The data analysis system of claim 7, wherein after the processor receives the treatment data corresponding to each of the candidate diagnostic codes, the processor generates cost data corresponding to each of the candidate diagnostic codes based on the corresponding treatment data or historical record, and each of the cost data is recorded in a cost field.

10. The data analysis system of claim 1, wherein the application model is based on a Bidirectional Encoder Representations from Transformers-Convolutional Neural Networks (BERT-CNN) implementation, the BERT-CNN determines a plurality of word vectors according to context of the content of the optimization report, the processor performs feature extraction based on a plurality of pre-defined word features in each layer of the BERT-CNN to extract the words;
wherein, after the word vectors pass through a classification layer of the BERT-CNN, the classification layer outputs the corresponding weights for each word vector, and the processor marks the words corresponding to the weights in different colors in the optimization report to generate the heat map;
wherein the processor is further used to generate a word cloud according to the weights.

11. A data analysis method, comprising:
displaying a user interface; wherein the user interface includes a plurality of medical information fields;
transmitting a part of contents of the plurality of medical information fields;
generating an optimization report using a processor based on the part of the contents of the medical information fields;
inputting the optimization report into an application model using the processor, wherein the application model outputs a plurality of diagnostic codes corresponding to the optimization report;
generating a heat map using the processor according to a plurality of weights corresponding to a plurality of words in the optimization report; and
displaying the heat map through a user interface using the processor.

12. The data analysis method of claim 11, further comprising:
displaying the user interface; wherein the user interface comprises the medical information fields;
transmitting the part of the contents of the medical information fields through a first transmission interface; and
receiving the part of the contents of the medical information fields;
wherein the medical information fields include a subjective field, a diagnosis observation field, a diagnosis assessment field, and a treatment plan field;
wherein the contents of the part of the fields includes the contents of the subjective field, the contents of the diagnosis observation field, and the contents of the diagnosis assessment field, as well as the rest of the text report of the patient within half a year.

13. The data analysis method of claim 12, further comprising:
performing a content optimization based on the part of the contents of the medical information fields through the processor to generate the optimization report.

14. The data analysis method of claim 13, wherein the content optimization includes using an abbreviation reduction Application Programming Interface (API) to change abbreviations in the part of the contents of the medical information fields to full names;
wherein the part of the contents of the medical information fields is automatically changed to correct text through a typo-correction suggestion application program interface, so as to automatically change a typo to the correct word or receive a corrected word that corrects the typo, to generate the optimization report.

15. The data analysis method of claim 11, wherein the application model outputs corresponding to the diagnostic codes;
wherein the diagnostic codes comply with a disease classification coding rule of the tenth edition of the International Classification of Disease (ICD);
wherein for multiple disease diagnosis and multiple predictions, the disease classification coding rule compiles the diagnostic codes corresponding to these disease diagnoses and the diagnostic codes for these predictions.

16. The data analysis method of claim 11, wherein the processor sorts the diagnostic codes corresponding to the weights according to the weights in descending order to generate a diagnosis code list.

17. The data analysis method of claim 16, wherein the processor selects a plurality of candidate diagnosis codes in the diagnosis code list, receives treatment data corresponding to each of the candidate diagnosis codes, and records the treatment data in a treatment plan field.

18. The data analysis method of claim 16, wherein the processor selects a plurality of candidate diagnostic codes in the diagnosis code list, based on a historical record, generates cost data corresponding to each of the candidate diagnostic codes, and records the cost data respectively in a cost field corresponding to the candidate diagnosis codes.

19. The data analysis method of claim 17, wherein after the processor receives the treatment data corresponding to each of the candidate diagnostic codes, the processor generates cost data corresponding to each of the candidate diagnostic codes based on the corresponding treatment data or historical record, and each of the cost data is recorded in a cost field.

20. The data analysis method of claim 11, wherein the application model is based on a Bidirectional Encoder Representations from Transformers-Convolutional Neural Networks (BERT-CNN) implementation, the BERT-CNN determines a plurality of word vectors according to the context of the content of the optimization report, the BERT-CNN determines a plurality of word vectors according to the context of the content of the optimization report, the processor performs feature extraction based on a plurality of pre-defined word features in each layer of the BERT-CNN to extract the words;
wherein, after the word vectors pass through a classification layer of the BERT-CNN, the classification layer outputs the corresponding weights for each word vector, and the processor marks the words corresponding to the weights in different colors in the optimization report to generate the heat map;
wherein the processor is further used to generate a word cloud according to the weights.
